(19) United States
(12) Patent Application Publication
(10) Pub. No.: US 2017/0258869 A1
(43) Pub. Date: Sep. 14, 2017

(54) Pharmaceutical Compositions for Colon-Specific Delivery

(71) Applicant: Gateway Pharmaceutical LLC, Chesterfield, MO (US)

(72) Inventor: Lianli Li, Chesterfield, MO (US)

(21) Appl. No.: 15/379,371

(22) Filed: Dec. 14, 2016

Related U.S. Application Data
(60) Provisional application No. 62/307,407, filed on Mar. 11, 2016.

(51) Int. Cl. A61K 38/14; A61K 9/20; A61K 9/48; A61K 9/50; A61K 31/606; A61K 31/4164

(52) U.S. Cl. CPC A61K 38/14; A61K 31/606; A61K 31/4164; A61K 9/4825; A61K 9/4808; A61K 9/5036; A61K 9/5026; A61K 9/2081; A61K 9/205; A61K 9/2054; A61K 9/2018; A61K 9/2027

(57) ABSTRACT

Disclosed are pharmaceutical particulates which release a pharmaceutical compound into the colon following oral administration. A particulate comprises a core comprising a pharmaceutical compound, an inner coating surrounding the core, wherein the inner coating comprises a pharmaceutically acceptable polysaccharide that is susceptible to enzymatic digestion by one or more enzymes present colonic microflora, and an outer coating surrounding the inner coating, wherein the outer coating comprises a polymer which is stable at upper gastrointestinal pH but can dissolve at colon luminal pH in less than about 60 minutes. The core of a particulate can further comprise an excipient such as a diluent, a binder, a disintegrant, a lubricant, a glidant or a combination thereof. Particulates can comprise pharmaceutical compounds for treating colonic diseases such as C. difficile colitis, ulcerative colitis, and Crohn's disease.

### CROSS-REFERENCE TO PRIOR APPLICATION

This application claims benefit of and priority to U.S. Provisional Patent Application 62/307,407, filed on Mar. 11, 2016. U.S. Provisional Patent Application 62/307,407 is hereby incorporated by reference in its entirety.

### INTRODUCTION

Pharmaceutical treatment of colon diseases, such as C. difficile Colitis, ulcerative colitis, and Crohn's disease, requires that the drug reach the colon in an effective form. Current pharmaceutical treatments include oral dosage forms and injections, however, these forms of dosage administration have several disadvantages relative to the present teachings. Non-targeted dosage forms frequently have side effects on the whole body. Additionally, non-targeted dosage forms may not effectively reach the infected location because they are released throughout the gastrointestinal tract.

Consequently, many different groups have attempted to develop formulations that target the colon. The majority of formulations include pH-dependent coatings. The jejunum of the small intestine can have a luminal pH of 5.0-6.0 and the ileum of the small intestine can have a luminal pH from 6.0-7.5 (Kumar, R. et al., Int. J. PharmTech Res., 2008, 1, 334-346). Colon turning pH can range from 6.0-7.6 (Kumar, R. et al., Int. J. PharmTech Res., 2008, 1, 334-346). Since the distal side of the small intestine can have a pH similar to that of the colon, existing formulations with pH-dependent coatings do not dissolve primarily at the colon.

U.S. Pat. No. 9,192,583 to Shah et at discloses a multiparticulate formulation for a volatile terpene-based active ingredient. A particulate of this disclosure includes a solid core containing the terpene-based active ingredient and a microcellulose excipient, a continuous proteinaceous subcoating over the core, and an enteric coating over the subcoating. This patent does not disclose the use of pectin or other polysaccharides. Additionally, the multiparticulate taught in this patent releases 50% of its terpene-based active ingredient in 1 hour with total release over 8 hours.

U.S. Pat. No. 6,224,910, to Ullah et al. teaches a dosage form with a polymethacrylate coating such as a EUDRAGIT® L (Evonik Corporation, Parsippany, N.J.) and an optional top coating of an anti-adherent, which can be hydrophobic material such as talc, magnesium stearate or fumed silica. This formulation is stable at pH 3, but releases at pH 4.5. This patent does not disclose the use of pectin or other polysaccharides.

U.S. Pat. No. 5,914,132 to Kelm et al. teaches a pharmaceutical dosage form with two pH dependent polymer coatings. This patent does not disclose the use of pectin or other polysaccharides.

Polysaccharides are used in formulations intended to target drugs to the colon. However, such formulations are slow-release formulas that do not provide drug release concentrated at the colon.

U.S. Pat. No. 9,237,760 to Ravishankar et al. discloses a pharmaceutical or neutraceutical core, surrounded by two controlling layers. The inner controlling layer surrounds the core and comprises cationic copolymers and anionic copolymers; the outer controlling layer comprises anionic copolymers. However, this patent does not disclose separate layers for copolymers and polysaccharides, nor does it disclose pectin. This patent also discloses an extended release formulation: complete release is described as occurring in “8 hours or less”.

Newton, A. M. J., et al. (Int. J. Appl. Res. Nat. Prod., 2012, 5, 1-16) compared the dissolution of mesalamine tablets that were coated with either a mixture of pectin and hydroxypropylmethylcellulose (HPMC) EUDRAGIT® L100 polymethacrylate (Evonik Corporation, Parsippany, N.J.) to form a slow release formulation. This reference does not disclose a dosage form coated with both pectin and a polymethacrylate.

Auriemma, G., et al (Carbohydrate Polymers, 2013, 92, 367-373) describes a multi-particulate drug delivery system produced by a prilling technique in combination with an enteric coating. This reference describes a formulation of an active drug (piroxicam) mixed with a Zn⁺-pectin, and EUDRAGIT® S100 polymethacrylate coating. However, this reference does not disclose using pectin as part of a coating of a microparticulate. This reference also discloses approximately 50-65% of dnig release in simulated gastric fluid for their formulation.

U.S. Pat. No. 9,023,368 to Basit et al. describes a slow release enteric coating that is a mixture of high polysaccharide starches (amylose, amylopectin) and polymers such as EUDRAGIT® S polymethacrylate, but explicitly excludes multi-layer dosage forms.

Milojevic, S. et al. (J. Controlled Release, 1996, 38, 75-94) describes layered coatings of glassy amylose with non-pH dependent polymer EUDRAGIT® RS/RL 30D polymethacrylate dispersions in various formulations (plasticized with PEG 300), but were unable to achieve colon-targeted delivery using such formulations. Consequently, this reference recommends mixtures that include ETHOCEL® ethylcellulose (The Dow Chemical Company, Midland, Mich.).

U.S. Pat. No. 6,228,396 to Watts describes a starch capsule intended for targeted release of a drug in the colon. The capsule has a coating of a copolymer of methacrylic acid and methyl methacrylic that dissolves at a pH of 5 or higher. While this patent discloses particulates such as mini-tablets for use in filling a starch capsule, it does not disclose the application of a pH-dependent coating directly to a particulate. This patent discloses polysaccharides including pectin as potential capsule-coating materials that promote release in the colon, but does not describe a particulate comprising pectin or a particulate having a pH-dependent outer coating. Furthermore, the formulation also serves as a slow release formulation: the drug in a capsule described in the patent is not released for over an hour after transfer to a neutral pH buffer.

There is thus an unmet need for a pharmaceutical formulation which, following oral administration, rapidly releases a pharmaceutical compound upon reaching the colon.

### SUMMARY

The present inventor has developed drug delivery systems which include mil compositions that can be used to target drugs to the colon, and methods of administering these compositions. In various embodiments, an oral composition of the present teachings can be a particulate which includes a) a core comprising an active pharmaceutical compound or salt thereof, b) an inner coating surrounding the core, and c) an outer coating which surrounds the inner coating. In various configurations, a particulate of the present teachings can be a pellet, a bead or a minitablet.

In various configurations, a core of a particulate of the present teachings can comprise any of a variety of active pharmaceutical compounds, such as, for example and without limitation, an antibiotic or an anti-inflammatory drug. Non-limiting examples of pharmaceutical compounds that can comprise a core of an oral composition of the present teachings include metronidazole, mesalamine, budesonide, mesalazine, vancomycin, fidaxomicin, rifaximin, ciprofloxacin, sulfasalazine, olsalazine, balsalazide, prednisone, hydrocortisone, infliximab, adalimumab, azathioprine, esomeprazole, mercaptopurine, vedolizumab, ciprofloxacin, golimumab, loperamide, methotrexate and pantoprazole.

In some configurations, an oral composition of the present teachings can comprise a pharmaceutically acceptable amount of a pharmaceutical compound. In some configurations, the amount of an active pharmaceutical ingredient in a particulate can range from 1 mg up to 1000 mg, or greater, 100-900 mg, 200-800 mg, or 250-750 mg. In various configurations, the weight of an active pharmaceutical ingredient in a particulate of the present teachings can be from 5 mg up to 15 mg, or front about 5 mg up to about 15 mg. In various configurations, a pharmaceutically acceptable amount of a pharmaceutical compound can be, for example and without limitation, 1 mg, about 1 mg, 2 mg, about 2 mg, 3 mg, about 3 mg, 4 mg, about 4 mg, 5 mg, about 5 mg, 6 mg, about 6 mg, 7 mg, about 2 mg, 15 mg, about 8 mg, 9 mg, about 9 mg, 10 mg, about 10 mg, 15 mg, about 15 mg, 20 mg, about 20 mg, 25 mg, about 2.5 mg, 30 mg, about 30 mg, 35 mg, about 35 mg, 40 mg, about 40 mg, 45 mg, about 45 mg, 50 mg, about 50 mg, 75 mg, about 75 mg, 100 mg, about 100 mg, 150 mg, about 150 mg, 200 mg, about 200 mg, 2.50 mg, about 250 mg, 300 mg, about 300 mg, 350 mg, about 350 mg, 400 mg, about 400 mg, 450 mg, about 450 mg, 500 mg, about 500 mg, 550 mg, about 550 mg, 600 mg, about 600 mg, 650 mg, about 650 mg, 700 mg, about 700 mg, 750 mg, about 750 mg, 800 mg, about 800 mg, 850 mg, about 850 mg, 900 mg, about 900 mg, 950 mg, about 950 mg, 1000 mg, or about 1000 mg of a pharmaceutical compound. In some configurations of an oral composition of the present teachings, a pharmaceutically acceptable amount of a pharmaceutical compound can be 250 mg, 500 mg or 750 mg metronidazole.

In some configurations, a core in accordance with the present teachings can further comprise an excipient, such that the core can comprise, consist essentially of, or consist of a mixture of an active pharmaceutical compound or a salt thereof, and an excipient. In some configurations, an excipient can be or can comprise a diluent, a binder, a disintegrant, a lubricant, a glidant or a combination thereof. In some configurations, an excipient can be a microcrystalline cellulose, a polyvinylpyrrolidone, a sodium starch glycolate, a lactose monohydrate, a cross-linked polyvinyl N-pyrrolidone, a hydroxypropyl cellulose, a hydroxypropylmethylcellulose, a croscarmellose sodium, a crospovidone, silicon dioxide, magnesium stearate or a combination thereof.

In some configurations, the weight ratio of drug to excipient in a core of the present teachings can range from 1:20 to 20:1, such as 1:20, about 1:20, 1:10, about 1:10, 3:20, about 3:30, 1:5, about 1:5, 1:4, about 1:4, 3:10, about 3:10, 7:20, about 7:20, 2:5, about 2:5, 9:20, about 9:20, 1:2, about 1:2, 11:20, about 11:20, 3:5, about 3:5, 13:20, about 13:20, 7:10, about 7:10, 3:4, about 3:4, 4:5, about 4:5, 17:20, about 17:20, 9:10, about 9:10, 19:20, about 19:20, 1:1, about 1:1, 20:19, about 10:19, 10:9, about 10:9, 20:17, about 20:17, 5:4, about 5:4, 4:3, about 4:3, 10:7, about 10:7, 20:13, about 10:13, 5:3, about 5:3, 20:11, about 10:11, 2:1, about 2:1, 20:9, about 20:9, 5:2, about 5:2, 20:7, 20:7, 10:3, about 10:3, 4:1, about 4:1, 5:1, about 5:1, 20:3, about 10:3, 10:1, about 10:1, 20:1, or about 20:1.

In some configurations, a core in accordance with the present teachings can comprise a pharmaceutical compound such as, without limitation, metronidazole, mesalamine, budesonide, mesalazine, vancomycin, fidaxomicin, rifaximin, ciprofloxacin, sulfasalazine, olsalazine, balsalazide, prednisone, hydrocortisone, infliximab, adalimumab, azathioprine, esomeprazole, mercaptopurine, vedolizumab, ciprofloxacin, golimumab, loperamide, methotrexate, pantoprazole, a pharmaceutically acceptable salt thereof, or a combination thereof. In some configurations, the pharmaceutical compound can be metronidazole, or a pharmaceutically acceptable salt thereof.

In various configurations, an inner coating can comprise, consist essentially of, or consist of at least one pharmaceutically acceptable polysaccharide that is subject to enzymatic digestion by one or more enzymes present in colonic microflora such as colonic bacteria (see, e.g., Flint, H. J., et al., Gut Microbes 3:4, 289-306, 2012). In some configurations, the polysaccharide can be a polysaccharide that can be subject to hydrolysis by an enzyme produced by colonic microflora such as, for example, a pectinase. In some configurations, a polysaccharide comprised by an inner coating can be a pectin or another polysaccharide substrate of pectinase.

In some configurations, an inner coating of the present teachings can provide a seal coating of a core of the present teachings.

In some configurations, an inner coating of the present teachings can comprise, consist essentially of, or consist of a polysaccharide. In some configurations, the polysaccharide can be a pharmaceutically acceptable polysaccharide, and can be a pectinase substrate, or another polysaccharide subject to digestion by enzyme(s) produced by colonic micro-flora (Nugent, S. G., Gut 48: 571-577, 2001). In some configurations, the polysaccharide can be a pectin, an amylose, a guar gum, an inulin, a dextran, a chitosan, a chondroitin sulfate or a combination thereof. In some configurations, the polysaccharide can be a pectin. In some configurations, the pectin can be a high methylester (HM) pectin.

In various configurations, the weight of an inner coating of a particulate of the present teachings can be 1% relative to the weight of a core, about 1%, 2%, about 2%, 3%, about 3%, 4%, about 4%, 5%, about 5%, 6%, about 6%, 7%, about 7%, 8%, about 8%, 9%, about 9%, 10%, about 10%, 11%, about 11%, 12%, about 12%, 13%, about 13%, 14%, about 14%, 15%, about 15%, 16%, about 16%, 17%, about 17%, 18%, about 1S%, 19%, about 19%, 20%, or about 20% relative to the weight of a core.

In some configurations, an outer coating can comprise, consist essentially of, or consist of a polymer that is stable at low pH i.e., at pH≦6.0, but can dissolve at a pH>6.0. In various configurations, an outer coating can comprise, consist essentially of, or consist of a polymer that is stable in acidic conditions such as found in the stomach but can dissolve at a higher pH range such as a pH of the lumen of the colon. In various eonfigurations, an outer coating of an oral composition of the present teachings can be stable at stomach pH and upper intestine luminal pH, but can dissolve at colon luminal pH in 30 minutes or less, about 30 minutes or less, 40 minutes or less, about 40 minutes or less, 50 minutes or less, about 50 minutes or less, 60 minutes or less, or about 60 minutes or less.

In various configurations, an outer coating that can resist dissolution in a stomach environment but can dissolve in a colon environment can comprise, consist essentially of, or consist of a pharmaceutically acceptable, pH-sensitive polymer such as, without limitation, a polymethacrylate, a cellulose acetate phthalate (CAP), a cellulose acetate trimellitate (CAT), a hydroxypropylmethylcelhdose phthalate (HPMCP) or a combination thereof.

In some configurations, an outer coating can comprise, consist essentially of, or consist of a pharmaceutically acceptable polymer which can be stable at a pH range below pH 6.0 that includes stomach pH (pH 1.5-3.5) and upper intestinal luminal pH pH 4.0-5.0) but can dissolve at a pH range that includes distal ileum lumen pH and colon lumen pH (pH>6). In various configurations, an outer coating can dissolve at pH>6.0, up to about pH 7.7, at least pH 6, at least pH 6.5. about pH 6.5, at least pH 7, about pH 7, at least pH 7.5, about pH 7.5, or higher. In various configurations, a particulate of the present teachings can comprise an outer coating that does not dissolve at a pH less than 6.0 but can dissolve at pH≧6.0. In various configurations, the outer coating does not dissolve at a pH>6.0 for at least two hours, such as, for example, at a pH ranging from pH 1.0 to pH 6.0, but dissolves within about 60 minutes at pH>6.0, a pH≧6.1, pH≧6.2, a pH≧6.3, a pH≧6.4, a pH≧6.5, a pH≧6.6, a pH≧6.7, a pH≧6.8, a pH≧6.9, a pH≧7.0, a pH≧7.1, or a pH≧7.2. In some configurations, an outer coating can dissolve at pH≧7.0, in various configurations, an outer coating can be stable at pH 1, about pH 1, pH≦1.5, about pH 1.5, pH≦2, about pH 2, pH≦2.5, about pH 2.5, pH≦3, about pH 3, pH≦3.5, about pH 3.5, pH≦4, about 4, pH≦4.5, about 4.5, pH≦5, about pH 5, pH≦5.5, about pH 5.3, or ≦pH 6.0.

In some configurations, a particulate of the present teachings can have an outer coating that is proportionally 3% of total particulate weight, about 3%, 4%, about 4%, 5%, about 5%, 6%, about 6%, 7%, about 7%, 8% about 8%, 9%, about 9%, 10%, about 10%, 11%, about 11%, 12%, about 12%, 13%, about 13%, 14%, about 14%, 15%, about 15%, 16%, about 16%, 17%, about 17%, 18%, about 18%, 19%, about 19%, 20%, about 20%, 21%, about 21%, 22%, about 22%, 23%, about 23%, 24%, about 24%, 25%, about 25%, 26%, about 26%, 27%, about 27%, 28%, about 28%, 29%, about 29%, 30%, or about 30% of total pimlieulate weight.

In some configurations, a prticulate in accordance with the present teachings can comprise an outer coating that can comprise, consist essentially of, or consist of a pharmaceutically acceptable, pH-sensitive polymer such as, without limitation, a polymethacrylate. In various configurations, the polymethacrylate can be, for example and without limitation, a polymer such as poly(methyl acrylate-co-methyl methacrylate-co-methacrylic acid) 7:3:1 (e.g., EUDRAGIT® FS 30 D) (Evonik Corporation, Parsippany, N.J.), poly(methacrylic acid-co-methyl methacrylate) 1:1 (e.g., EUDRAGIT® L100), poiy(methacrylic acid-co-methyl methacrylate) 1:2 (e.g., EUDRAGIT® S100), singly or in combination with poly(methacrylic acid-co-ethyl acrylate) 1:1 (eg, EUDRAGIT® L 30 D-55).

In some configurations, an outer coating of a particulate of the present teachings can further comprise one or more pharmaceutically acceptable coating additives, such as, without limitation, an anti-tacking agent, a plasticizer, a stabilizer or a combination thereof. In various configurations, a coating additive can be, for example and without limitation, a PLASACRYL™ such as PLASACRYL™ T20 (Evonik Corporation, Parsippany, N.J.), triethyl citrate, acetyl tributyl citrate, acetyl triethyl citrate, diacetylated monoglycerides, dibutyl sebacate, diethyl phthalate, triacetin, tributyl citrate, glycerin, a polyethylene glycol, a polyethylene glycol monomethyl ether, a propylene glycol, sorbitol sorbitan, TiO₂, talc, glycerol monostearate, a polysorbate, or a combination thereof.

In some embodiments, a pharmaceutical dosage form of the present teachings can comprise a ptundity of particulates, each particulate comprising a core comprising a pharmaceutical compound, a polysaccharide inner coating somounding the core, and an outer coating comprising a polymer such as a polymethtterylate, a cellulose acetate phthalate (CAP), a cellulose acetate trirnellitate (CAT), a hydroxypropylmethylcellulose phthalate (HPMCP) or a combination thereof, wherein the outer coating surrounds the inner coating. In some configurations, a shell can encapsulate a plurality of particulates.

In some configurations, a pharmaceutical dosage form of the present teachings can be a tablet which can comprise a plurality of particulates plus additional excipient material such as a hinder, a colorant, a disintegrant, a lubricant, a glidant, a flavoring, a preservative, diluent or a combination thereof.

In some configurations, a pharmaceutical dosage form of the present teachings can comprise a plurality of particulates and a shell encapsulating the plurality of particulates. In some configurations, a shell can comprise a pharmaceutically acceptable material such as, without limitation, a gelatin, a hydroxypropylmethyl cellulose or a combination thereof.

In some configurations, an oral composition of the present teachings such as a tablet or capsule can comprise a plurality of particulates, each particulate comprising an inner coating comprising a pharmaceutically acceptable polysaccharide. In some configurations, the polysaccharide can be a pectinase substrate. In various configuration, the polysaccharide can be, such as but without limitation, a pectin, a guar gum, an amylose, an inulin, a dextran, a chitosan, a chondroitin sulfate or a combination thereof. In various configurations, the polysaccharide can be a pectin. In some configurations, the pectin can be HM grade pectin.

In some configurations, the outer coating of a particulate of the present teachings can comprise a pharmaceutically acceptable polymer such as a polymethaelylate. In some configurations, the polymethacrylate can be poly(methyl acrylate-co-methyl methacrylate-co-methacrylic acid) 7:3:1 or a mixture comprising poly(methyl acrylatc-co-methyl methacrylate-co-methacrylic acid) 7:3:1 and poly(methacrylic acid-co-ethyl acrylate) 1:1.

In some configurations, an oral composition of the present teachings can comprise an outer coating comprising one or more coating additives. In some configurations a coating additive can be an anti-tacking agent, a plasticizer and a stabilizer. In various configurations, a coating additive can be, without limitation, PLASACRYL™ (Evonik Corporation, Parsippany, N.J.), triethyl citrate, acetyl tributyl citrate, acetyl triethyl citrate, diacetylated monoglycerides, dibutyl sebacate, diethyl phthalate, triacetin, tributyl citrate, glycerin, polyethylene glycols, polyethylene glycol monomethyl ether, propylene glycol, sorbitol sorbitan, TiO₂, talc, glycerol monostearate, or a polysorbate.

In some configurations, an oral composition of the present teachings can comprise an outer coating that does not dissolve at a pH less than 6.0 and dissolves at pH 6.0. In some configurations, the outer coating can dissolve at pH>6, pH 6.5, about pH 6.5, pH 7, about pH 7, pH 7.5 or about pH 7.5. In some configurations the outer coating does not dissolve at a pH less than 6.0 in at least 120 minutes and dissolves at pH≧6.0 in 30 minutes or less.

In some configurations, a particulate of an oral composition of the present teachings can further comprise an excipient such as, but without limitation, microcrystalline cellulose, hydroxypropyl cellulose, croscarmellose sodium, magnesium stearate or a combination thereof. In various configurations, a particulate can comprise about 15% microcrystalline cellulose, about 5% hydroxypropyl cellulose, about 3% croscarmellose sodium and about 0.3% magnesium stearate.

In some configurations, an oral composition of the present teachings can comprise an outer coating comprising, consisting essentially of, or consisting of poly(methyl acrylate-co-methyl methacrylate-co-methacrylic acid) 7:3:1 and PlasACRYL™ T20. In various configurations, the poly(methyl acrylate-co-methyl methacrylate-co-methacrylic acid) 7:3:1:PlasACRYL™ T20 ratio can be 10:1. In various configurations the poly(methyl acrylate-co-methyl methacrylate-co-methacrylic acid) 7:3:1:PlasACRYL™ T20 ratio can be 1:1, 2:1, 3:1, 4:1, 5:1, 6:1, 7:1, 8:1, 9:1, or 10:1. In various configurations, the outer coating can comprise poly(methyl acrylate-co-methyl methacrylate-co-methacrylic acid) 7:3:1 and PlasACRYL™ T20 in a 10:1 ratio. In various configurations, the ratio of poly(methyl acrylate-co-methyl methacrylate-co-methacrylic acid) 7:3:1 and PlasACRYL™ T20 can be 1:1, 2:1, 3:1, 4:1, 5:1, 6:1, 7:1, 8:1, 9:1, or 10:1. In various configurations, the ratio of poly(methyl acrylate-co-methyl methacrylate-co-methacrylic acid) 7:3:1 and PlasACRYL™ T20 can be, without limitation, 20:1, 19:1, 18:1, 17:1, 16:1, 15:1, 14:1, 13:1, 12:1, 11:1, 10:1, 9:1, 8:1, 7:1, 6:1, 17:3, 5:1, 4:1, 3:1, 7:3, 2:1, 13:7, 3:2, 11:9, or 1:1 (w/w).

In various configurations, the outer coating can comprise, consist essentially of or consist of poly(methyl acrylate-co-methyl methacrylate-co-methacrylic acid) 7:3:1 and poly(methacrylic acid-co-ethyl acrylate) 1:1 (w/w). In various configurations, the outer coating can comprise poly(methyl acrylate-co-methyl methacrylate-co-methacrylic acid) 7:3:1:poly(methacrylic acid-co-ethyl acrylate) 1:1 in a 6:1 ratio (w/w). In various configurations, the poly(methyl acrylate co-methyl methacrylate-co-methacrylic acid) 7:3:1:poly(methacrylic acid-co-ethyl acrylate) ratio can be 1:1, 2:1, 3:1, 4:1, 5:1, 6:1, 7:1, 9:1 or 10:1 (w/w). In various configurations, the poly(methyl acrylate-co-methyl methacrylate-co-methacrylic acid) 7:3:1:poly(methacrylic acid-co-ethyl acrylate) 1:1 weight ratio can range from 99:1 to 1:1, such as, without limitation, 95:5, 90:10, 85:15, 80:20, 75:25, 70:30, 65:35, 60:40, 55:45, or 50:50 (% w/w). In various configurations, the poly(methyl acrylate-co-methyl methacrylate-co-methacrylic acid) 7:3:1:poly(methacrylic acid-co-ethyl acrylate) 1:1 ratio can be 85:15 (% w/w), or about 85:15.

In some configurations, the outer coating can comprise triethyl citrate, titanium dioxide, and poly(methyl acrylate-co-methyl methacrylate-co-methacrylic acid) 7:3:1 and poly(methacrylic acid-co-ethyl acrylate) 1:1. In various configurations, the poly(methyl acrylate-co-methyl methacrylate-co-methacrylic acid) 7:3:1:poly(methacrylic acid-co-ethyl acrylate) 1:1 weight ratio can be 6:1. In various configurations, the poly(methyl acrylate-co-methyl methacrylate-co-methacrylic acid) 7:3:1:poly(methacrylic acid-co-ethyl acrylate) 1:1 weight ratio can range from 1:1 up to 10:1, and can be, for example, 1:1, 2:1, 3:1, 4:1, 5:1, 6:1, 7:1, 8:1, 9:1 or 10:1. In various configurations, the poly(methyl acrylate-co-methyl methacrylate-co-methacrylic acid) 7:3:1:poly(methacrylic acid-co-ethyl acrylate) 1:1 ratio can be 95:5, 90:10, 85:15, 80:20, 75:25, 70:30, 65:35, 60:40, 55:45, or 50:50 (% w/w). In various configurations, the poly(methyl acrylate-co-methyl methacrylate-co-methacrylic acid) 7:3:1:poly(methacrylic acid-co-ethyl acrylate) 1:1 weight ratio can range from 20:1 to 1:1, and can be, for example, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1 or 1:1. In various configurations, the poly(methyl acrylate-co-methyl methacrylate-co-methacrylic acid) 7:3:1:poly(methacrylic acid-co-ethyl acrylate) 1:1 ratio can be about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1 or about 1:1. In various configurations, the poly(methyl acrylate-co-methyl methacrylate-co-methacrylic acid) 7:3:1:poly(methacrylic acid-co-ethyl acrylate) 1:1 weight ratio can be 85:15 (% w/w).

In various configurations, an oral composition of the present teachings can comprise an inner coating comprising pectin and titanium dioxide. In various configurations, the pectin:titanium dioxide ratio can be 10:1. In various configurations, the pectin:titanium dioxide ratio can be 1:1, about 1:1, 2:1, about 2:1, 3:1, about 3:1, 4:1, about 4:1, 5:1, about 5:1, 6:1, about 6:1, 7:1, about 7:1, 8:1, about 8:1, 9:1, about 9:1, 10:1, about 10:1, or greater.

In some embodiments, an oral composition of the present teachings can be a capsule comprising a plurality of particulates, and a capsule shelf. In various configurations, each particulate can comprise a core comprising a pharmaceutical compound, a pectinase-sensitive inner coating sin-rounding the core, and an outer coating comprising a polymer selected from the group consisting of a polymethacrylate, cellulose acetate phthalate (CAP), a cellulose acetate trimellitate (CAT), a hydroxypropylmethylcellulose phthalate (HPMCP) and a combination thereof wherein the outer coating encapsulates the inner coating. In some configurations, the total amount of the pharmaceutical compound in a capsule can be from 200 to 800 mg metronidazole, such as, for example, 250, 500 or 750 mg metronidazole. In some configurations, each particulate can further comprise one or more excipients such as, without limitation, microcrystalline cellulose, hydroxypropyl cellulose, croscarmellose sodium and magnesium stearate. In some configurations, the inner coating can comprise pectin, and can further comprise titanium dioxide. In some configurations, the ratio of pectin: titanium dioxide can range from 5:1 to 20:1, such as 5:1, 6:1, 7:1, 8:1, 9:1, 10:1, 11:1, 12:1, 13:1, 14:1, 15:1, 16:1, 17:1, 18:1, 19:1 or 20:1 (w/w). In some configurations, the ratio of poly(methyl acrylate-co-methyl methacrylate-co-methacrylic acid) 7:3:1:PlasACRYL™ T20 can range from 5:1 to 20:1, such as 5:1, 6:1, 7:1, 8:1, 9:1, 10:1, 11:1, 12:1, 13:1, 14:1, 15:1, 16:1, 17:1, 18:1, 19:1 or 20:1 (w/w).

In some embodiments, a capsule of the present teachings can comprise: a plurality of particulates, each particulate can comprise a core comprising metronidazole, microcrystalline cellulose, hydroxypropyl cellulose, croscarmellose sodium and magnesium stearate wherein the total amount of metronidazole in the capsule can be 500 mg; an inner coating comprising pectin and titanium dioxide in a 10:1 ratio wherein the inner coating surrounds the core and an outer coating comprising poly(methyl acrylate-co-methyl methacrylate-co-methacrylic acid) 7:3:1 and poly(methacrylic acid-co-ethyl acrylate) 1:1 in a ratio of 85:15 (% w/w) wherein the outer coating encapsulates the inner coating; and wherein a shell encapsulates the plurality of particulates.

In some embodiments, a pharmaceutical capsule in accordance with the present teachings can comprise a plurality of particulates encased in a shell, wherein each particulate comprises: a core comprising metronidazole, microcrystalline cellulose, hydroxypropyl cellulose, croscarmellose sodium and magnesium stearate; an inner coating comprising pectin and titanium dioxide in a 10:1 ratio; and an outer coating comprising poly(methyl acrylate-co-methyl methacrylate-co-methacrylic acid) 7:3:1 and PlasACRYL™ T20 in a 10:1 ratio. In some configurations, a pharmaceutical capsule can comprise 500 mg metronidazole.

In some embodiments, a method of treating a colon disease can comprise administering to a subject in need thereof a pharmaceutical dosage form in accordance with the present teachings. In some configurations, the colon disease can be an inflammatory bowel disease such as C. difficile colitis, ulcerative colitis, or Crohn's disease. In some configurations, the pharmaceutical compound of the administered pharmaceutical dosage form can be metronidazole, mesalamine, budesonide, mesalazine, vancomycin, fidaxomicin, rifaximin, ciprofloxacin, sulfasalazine, olsalazine, balsalazide, prednisone, hydrocortisone, infliximab, adalimumab, azathioprine, esomeprazole, mercaptopurine, vedolizumab, ciprofloxacin, golimumab, loperamide, methotrexate and pantoprazole, a pharmaceutically acceptable salt thereof, or a combination thereof. In various configurations, the core can comprise metronidazole or a pharmaceutically acceptable salt thereof. In various configurations, pharmaceutical dosage form can have 500 mg of metronidazole. In some configurations, an administered pharmaceutical dosage form can comprise a plurality of particulates, each particulate comprising a core comprising a pharmaceutical compound; a pectinase-sensitive inner coating surrounding the core; and an outer coating comprising a polymer selected from the group consisting of a polymethacrylate, cellulose acetate phthalate (CAP), a cellulose acetate trimellitate (CAT), a hydroxypropylmethylcellulose phthalate (HPMCP) and a combination thereof, wherein the outer coating surrounds the inner coating; and wherein a shell encapsulates the plurality of particulates. In some configurations, the total amount of the pharmaceutical compound in the capsule can be 500 mg metronidazole; each particulate can further comprise microcrystalline cellulose, hydroxypropyl cellulose, croscarmellose sodium and magnesium stearate; the inner coating can comprise pectin and titanium dioxide in a 10:1 ratio; and the outer coating can comprise poly(methyl acrylate-co-methyl methacrylate-co-methacrylic acid) 7:3:1 and PlasACRYL™ T20 in a 10:1 ratio.

### BRIEF DESCRIPTION OF THE DRAWINGS

FIG. 1 is a schematic diagram of a particulate of the present teachings.

FIG. 2 is a schematic diagram of a capsule filled with particulates of the present teachings.

FIG. 3 is a schematic diagram of a tablet pressed from particulates of the present teachings and an excipient.

FIG. 4 illustrates dissolution testing at pH 7.0 in a basket apparatus at 50 rpm of cores described in example 3, with or without a seal (inner) coating of hydroxypropylmethylcellulose.

FIG. 5 illustrates dissolution profiles at pH 7.0, in a paddle apparatus at 50 rpm, of particulates having hydroxypropylmethylcellulose inner coatings and a polymethacrylate outer coatings.

FIG. 6 illustrates dissolution profiles at pH 7.0, in a paddle apparatus at 75 rpm, of particulates having a pectin inner coating and a polymethacrylate outer coating in the presence or absence of pectinase.

FIG. 7 illustrates the dissolution profiles at pH 6.0, in a paddle apparatus at 75 rpm, of particulates having inner coatings of pectin or hydroxypropylmethylcellulose, and a polymethacrylate outer coating.

FIG. 8 illustrates slow rate of drug dissolution at pH 6.0 of a mesalamine formulation of the present teachings.

FIG. 9 illustrates rapid rate of drug dissolution at pH 7.4 of a mesalamine &mutilation of the present teachings.

FIG. 10 illustrates that pectinase enhances the rate of drug dissolution of a mesalamine formulation of the present teachings.

### DETAILED DESCRIPTION

The present teachings include descriptions that are not intended to limit the scope of any claim. The examples and methods are provided to further illustrate the present teachings. Those of skill in the art, in light of the present disclosure, will appreciate that many changes can be made in the specific embodiments that are disclosed and still obtain a like or similar result without departing from the spirit and scope of the present teachings. As used herein, the singular forms “a”, “an” and “the” are intended to include the plural forms as well, unless the context indicates otherwise.

Methods and compositions described herein utilize laboratory techniques well known to skilled artisans. Methods of administration of pharmaceuticals and dosage regimes, can be determined according to standard principles of pharmacology well known skilled artisans, using methods provided by standard reference texts such as Remington: the Science and Practice of Pharmacy (Alfonso R. Gennaro ed. 19th ed. 1995); Hardman, J. G., et al., Goodman & Gilman's The Pharmacological Basis of Therapeutics, Ninth Edition, McGraw-Hill, 1996, Augsburger, L. L., et al. Pharmaceutical Dosage Forms—Tablets, CRC Press, 2008, Thakur, V. K., et al. Handbook of Polymers for Pharmaceutical Technologies, Biodegradable Polymers, John Wiley & Sons, 2015 and Rowe, R. C., et al., Handbook of Pharmaceutical Excipients, Pharmaceutical Press, 2012. All publications cited herein are incorporated by reference, each in its entirety.

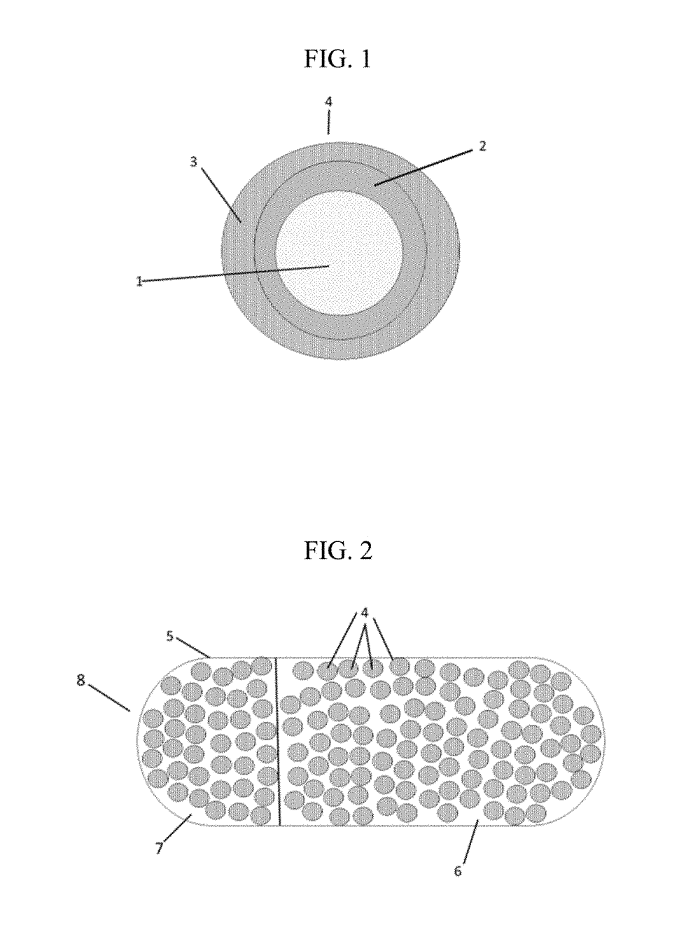

As shown in FIG. 1, a particulate (4) of the present teachings can comprise, consist essentially of or consist of a core (1) which is surrounded by an inner coating (2) which comprises, consists of, or consists essentially of a polysaccharide, and an outer coating (3) surrounding the inner coating, which comprises, consists essentially of, or consists of a pH-dependent polymer. Additional coatings or layers, such as, but without limitation, cosmetic coatings or non-reactive coatings, can also separate any of the layers or be used to further coat a complete particulate.

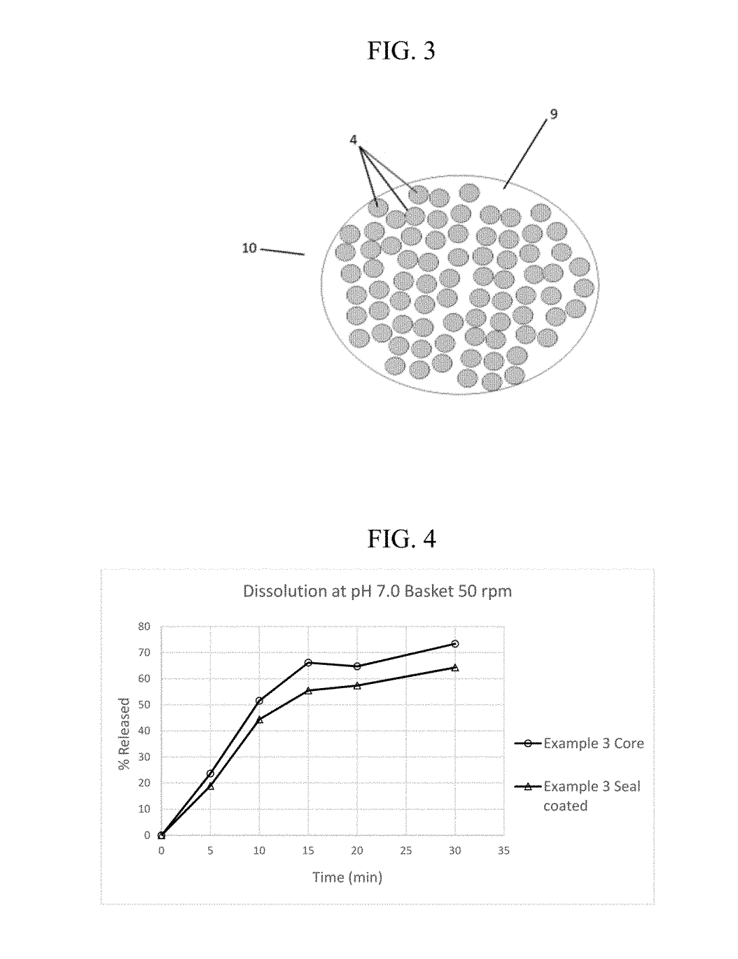

A particulate of the present teachings can comprise a single oral dosage form, or can be part of a multi particulate that is packaged into a larger oral dosage form. As illustrated in FIG. 2, a gelatinous capsule shell (5) can contain a plurality of particulates (4). A capsule (8) can include a body (6) and a cap (7). Upon oral administration, a gelatinous capsule shell can dissolve in the stomach and the plurality of particulates can then proceed to the colon. Alternately, as shown in FIG. 3, a plurality of particulates (4) can be combined with one or more excipients (9) and pressed into a tablet (10). In various configurations, a tablet can be further coated with a coating, such as, without limitation, a cosmetic coating.

As used herein, a “particulate” can be a bead, a pellet, or a mini-tablet, and can be a portion of a larger dosage form. A particulate, bead, or minitablet comprises a core, an inner coating, and an outer coating in accordance with the present teachings.

In various configurations, a particulate can be, without limitation, spherical or cylindrical in shape. In some configurations, a particulate can comprise from 1-10 mg of a pharmaceutical compound such as, for example, metronidazole. In some configurations, a particulate can have a diameter of from about 0.3 mm up to about 5 mm. In various configurations, the total weight of a particulate can range from about 1 mg up to about 25 mg.

In various configurations, a dosage form can be comprised of a plurality of particulates. A dosage form comprising a plurality of particulates can have, for example, from 250 mg-1000 mg, for example 500 mg of a pharmaceutical compound such as metronidazole.

As used herein, a core comprises a pharmaceutically active substance, and can further comprise one or more excipients in accordance with the present teachings.

As used herein, an inner coating comprises a polysaccharide that is sensitive to digestion by enzymes present in the lumen of the colon, in particular hydrolases harbored by colonic microflora. An inner coating can further comprise coating additives in accordance with the present teachings.

As used herein, an outer coating comprises a pH-dependent polymer that is stable at pH≦6.0, but dissolves at pH>6.0.

As used herein, a seal-coated core is a core coated with an inner coating.

Polysaccharides of the present teachings include polysaccharides that are subject to hydrolysis by enzymes of colonic microorganisms. Polysaccharides of the present teachings include, but are not limited to, amylose, arabinoga lactose, chitosan, cyclodextrins, chondroitin sulfate, pectin, dextran, guar gum, xylan and inulin.

Pectins are anionic polysaccharides extracted from plant primary cell walls. Pharmaceutical grade pectin is available under a variety of tradenames, e.g., GENU® (CP Kelco, Atlanta, Ga.). Pectin is commercially available in three grades depending on the degree of esterification: high methylester (HM), conventional low methylester pectin (LMC), and low methylester amidated (LMA).

Outer coating. As used herein, an outer coating is a pH-dependent enteric coating that is stable at pH 1-6 (stomach) but dissolves at pH>6 (i.e., at a pH range found in the lumen of the colon). pH-dependent coatings can include, without limitation, a polymethacrylate, a cellulose acetate phthalate (CAP), a cellulose acetate trimellitate (CAT), a hydroxypropylmethylcellulose phthalate (HPMCP), polyvinyl acetate phthalate (COATERIC™, Colorcon Ltd., Harleysville, Pa.), polyvinyl acetate phthalate (SURETERIC™ Colorcon, Ltd.), and cellulose acetate phthalate (AQUATERIC™, FMC Corp., Philadelphia, Pa.)

Hydroxypropylmethylcellulose phthalate. Hydroxypropylmethylcellulose phthalate is available in several grades under different tradenames, such as, without limitation, hydroxypropyl methylcellulose phthalate HP50 (HPMCP-HP50) (USP/N F 220824), HP55 (HPMCP-HP55) (USP/NF type 200731) and HP55S (Shin Etsu Chemical, Tokyo, Japan).

Polymethacrylate. Polymethacrylates include polymers that can be used in pharmaceutical coatings. Polymethacrylates are available under several trade names such as, for example and without limitation, EUDRAGIT® (Evonik Corporation, Parsippany, N.J.). Several polymethacrylates available under the tradename EUDRAGIT® can be used in a particulate of the present teachings, such as and without limitation, poly(methacrylic acid-co-ethyl acrylate) (L 30 D-55, L 100-55), poly(methacrylic acid-co-methyl methacrylate) 1:1 (L100, L 12,5), poly(methacrylic acid-co-methyl methacrylate) 1:2 (S100, S 12,5), and poly(methyl acrylate-co-methyl methacrylate-co-methacrylic acid) 7:3:1 (FS 30 D). Poly(methyl acrylate-co-methyl methacrylate-co-methacrylic acid) 7:3:1 is available in a 30% aqueous dispersion under the trade name EUDRAGIT® FS 30 D and dissolves at pH 7.0. Poly(methacrylic acid-co-ethyl acrylate) 1:1 is available as a 30% aqueous dispersion under the trade name EUDRAGIT® L-30-D-55 and dissolves at pH 5.5. Combinations of two polymethacrylates can be used to form coatings that dissolve at different pH levels; the relative amounts of polymethacrylates as well as coating additives can be adjusted to modify the pH stability of a coating.

Coating additives. A variety of materials can be added to the inner coating or the outer coating, such as a stabilizer, a plasticizer, and/or an anti-tacking agent. In some configurations, a stabilizer can be an emulsifier such as, for example, polysorbate 80. In some configurations, a plasticizer can be, for example, acetyl tributyl citrate, acetyl triethyl citrate, castor oil, diacetylate monoglycerides, dibutyl sebacate, diethyl phthalate, glycerin, glycerol, polyethylene glycols, polyethylene glycol monomethyl ether, polyvinylpyrrolidone, propylene glycol, sorbitol, sorbitan solution, triacetin, tributyl citrate, and triethyl citrate. In some configuration, an and-tacking agent can be, for example, colloidal silicon dioxide, fumed silica glycerol monostearate (GMS), magnesium stearate or talc. Commercially available coating additives can contain any of the foregoing types of coating additives in combination. For example, and without limitation, additives available under the tradename PLASACRYL™ (Evonik Corporation, Parsippany, N.J.), which contains a stabilizer, a plasticizer and an anti-tacking agent. PLASACRYL™ T20 can be used with polymethacrylates EUDRAGIT® FS 30 D and EUDRAGIT® L-30-D-55 (Evonik Corporation, Parsippany, N.J.). Other coating additives can include an opacifier or pigment, such as and without limitation, titanium dioxide (TiO₂).

Excipients. Non-limiting examples of excipients include microcrystalline cellulose, polyvinylpyrrolidone, hydroxypropylcellulose. Polyvinylpyrrolidone is available under several different grades, such as, for example and without limitation, K 15, K 25, K 30, and K 90. Hydroxypropylcellulose is available from various suppliers under a variety of trade names, such as KLUCEL™ (Ashland Inc., Covington, Ky.) HF Pharm, HXF Pharm, MF Pharm, MXF Pharm, GF Pharm, GXF Pharm, JF Pharm, JXF Pharm, LF Pharm, JXF Pharm, LF Pharm, LXF Pharm, EF Pharm, EXF Pharm, ELP Pharm; Nisso HPC (Nisso America Inc., New York, N.Y.) SSL, SL, L, and M.

Diluents. Non-limiting examples of diluents include microcrystalline cellulose, lactose monohydrate, lactose anhydrous, a starch such as maize starch, wheat starch, potato starch, or pregelatinized starch, a sugar such as sorbitol, mannitol, xylitol, dextrose, sucrose, or fructose, kaolin, calcium phosphate, calcium sulfate, and calcium carbonate. Non-limiting examples of microcrystalline cellulose include AVICEL® PH-101, PH-102, PH-103, PH-105, pH-112, PH-113, PH-200, PH-301 and PH-302 (FMC Corporation, Philadelphia, Pa.); PHARMACEL® 101, 102, and 112 (DFE Pharma, Paramus, N.J.); and GRINDSTED® (Danisco, Madison, Wis.).

Binders. Non-limiting examples of binders include microcrystalline cellulose, hydroxypropyl cellulose, such as KLUCEL™ (Ashland Inc., Covington, Ky.) HF, HXF, MF, MXF, GF, GXF, JF, JXF, LF, JXF, LF, LXF, EF, EXF, ELP, hydroxypropyl methylcellulose (HPMC), polyvinylpyrrolidone, sodium carboxymethylcellulose, sucrose, liquid glucose, acacia, tragacanth, gelatin, starch paste, pregelatinized starch, alginic acid, cellulose, methyl cellulose, ethyl cellulose potassium alginate and sodium alginate.

Disintegrants. Non-limiting examples of disintegrants include pregelatinized starch, microcrystalline cellulose, croscarmellose sodium, crospovidone, and sodium starch glycolate.

Lubricants. Non-limiting examples of lubricants include magnesium stearate, calcium stearate, stearic acid, hydrogenated vegetable oil, mineral oil, polyethylene glycol, sodium lauryl sulfate, glycerol palmitostearate, glyceryl behenate, sodium benzoate, and sodium stearyl fumarate.

Glidant. Non-limiting examples of glidants include colloidal silicon dioxide, silicon dioxide and talc.

### EXAMPLES

Unless specifically presented in the past tense, an example can be a prophetic or an actual example.

### Example 1

This example illustrates the preparation of uncoated cores of the present teachings.

TABLE 1

| | | |
|---|---|---|
| Ingredient | mg/Dose | % |

Cores

| | | |
|---|---|---|
| Mesalamine | 250 | 68.5 |
| Microcrystalline cellulose | 75 | 20.5 |
| Avicel ® PH-101 | | |
| (FMC, Philadelphia, PA) | | |
| Polyvinylpyrrolidone | 20 | 5.5 |
| (Povidone K30, | | |
| Kollidon ®, BASF) | | |
| Sodium starch glycolate | 20 | 5.5 |
| Purified water* | q.s | — |
| Total weight (mg) | 365 | 100.0 |

*Added but not retained

To generate uncoated cores, mesalamine, microcrystalline cellulose, polyvinylpyrrolidone and sodium starch glycolate were mixed in proportions set out in Table 1 in a high shear granulator, then granulated with water to form wet granulation. The wet mass was extruded and spheronized into beads. The beads were then dried in a fluid bed to form drug-containing beads.

### Example 2

This example illustrates the preparation of uncoated cores that can be used for forming particulates of the present teachings.

TABLE 2

| | | |
|---|---|---|
| Ingredient | mg/Dose | % |

Cores

| | | |
|---|---|---|
| Vancomycin | 125 | 50.4 |
| Lactose monohydrate | 50 | 20.2 |
| Microcrystalline cellulose | 50 | 20.2 |
| Avicel ® PH-102 | | |
| (FMC, Philadelphia, PA) | | |
| Polyvinylpyrrolidone | 15 | 6.0 |
| (Povidone K30, | | |
| Kollidon ®, BASF) | | |
| Crospovidone | 8 | 3.2 |
| Purified water* | q.s | — |
| Total weight (mg) | 248 | 100.0 |

*Added but not retained

In this procedure, vancomycin, and excipients lactose monohydrate, microcrystalline cellulose, and crospovidone were mixed in proportions set out in Table 2 in a fluid bed equipped with a bottom rotor, then pelletized by spraying from the side with polyvinylpyrrolidone in a water binding solution. The resulting pellets were then dried in a fluid bed.

### Example 3

This example illustrates the generation of seal coated cores having a hydroxypropyl methylcellulose coating.

TABLE 3

| | | |
|---|---|---|
| Ingredient | mg/Dose | % |

Cores

| | | |
|---|---|---|
| Metronidazole | 500 | 76.3 |
| Microcrystalline cellulose | 100 | 15.3 |
| Avicel ® PH-102 | | |
| (FMC, Philadelphia, PA) | | |
| Hydroxypropyl cellulose EXF | 33 | 5.0 |
| Croscarmellose sodium | 20 | 3.1 |
| Magnesium stearate | 2 | 0.3 |
| Total weight (mg) | 655 | 100.0 |

Inner Coating

| | | |
|---|---|---|
| hydroxypropyl methylcellulose | 30 | 4.6 |
| (SPECTRABLEND ™ White, | | |
| Sensient ® Pharmaceutical, | | |
| St. Louis, MO) | | |
| Purified water* | q.s | — |
| Total weight (mg) | 685 | — |

*Added but not retained

Manufacturing Procedure:

To generate cores, metronidazole, microcrystalline cellulose, hydroxypropyl cellulose, croscarmellose sodium and magnesium stearate were mixed in a blender in proportions set out in Table 3. The mixture was then compressed into mini-tablets on a rotary tablet press fitted with multi-tip punches. The mini-tablets were then coated with HPMC-based seal coat (SPECTRABLEND™ White) using a perforated tablet coater.

### Example 4

This example illustrates a procedure for forming seal-coated cores having pectin for the inner coating.

TABLE 4

| | | |
|---|---|---|
| Ingredient | mg/Dose | % |

Cores

| | | |
|---|---|---|
| Metronidazole | 500 | 76.3 |
| Microcrystalline cellulose | 100 | 15.3 |
| Avicel ® PH-102 | | |
| (FMC, Philadelphia, PA) | | |
| Hydroxypropyl cellulose EXF | 33 | 5.0 |
| Croscarmellose sodium | 20 | 3.1 |
| Magnesium stearate | 2 | 0.3 |
| Total weight (mg) | 655 | 100.0 |

Inner Coating

| | | |
|---|---|---|
| Pectin | 30 | 5.0 |
| TiO₂ | 3 | 0.5 |
| Purified water* | q.s | — |
| Total weight (mg) | 685 | — |

*Added but not retained

Manufacturing Procedure:

To generate cores, metronidazole, microcrystalline cellulose, hydroxypropyl cellulose, croscarmellose sodium and magnesium stearate were mixed in a blender in amounts shown in Table 4 and then compressed into mini-tablets on a rotary tablet press fitted with multi-tip punches. The cores were then coated with pectin-based inner coating in amounts shown in Table 4 using a perforated tablet coater to form seal-coated cores.

### Example 5

This example illustrates the coating of HPMC seal-coated cores with a pH-dependent outer coating.

TABLE 5

| Ingredient | mg/Dose | % |
|---|---|---|
| Example 3 coated | 685 | 100.0 |

Outer Coat

| | | |
|---|---|---|
| EUDRAGIT ® FS 30 D | 62 | 9.1 |
| PLASACRYL ™ T20 | 6.2 | 0.9 |
| Purified water* | q.s | — |
| Total weight (mg) | 753.2 | — |

*Added but not retained

Seal-coated cores made in Example 3 were further coated with EUDRAGIT® FS 30 D and PLASACRYL™ T20 dispersion in amounts shown in Table 5 using a perforated tablet coater to add an outer coating.

### Example 6

This example illustrates the coating of HPMC seal-coated cores with a pH-dependent outer coating.

TABLE 6

| Ingredient | mg/Dose | % |
|---|---|---|
| Example 3 coated | 685 | 100.0 |

Outer Coat

| | | |
|---|---|---|
| EUDRAGIT ® FS 30 D | 36 | 5.3 |
| EUDRAGIT ® L 30 D-55 | 6 | 0.9 |
| Triethyl citrate | 3 | 0.4 |
| TiO₂ | 23 | 3.4 |
| Purified water* | q.s | — |
| Coated weight (mg) | 753 | — |

*Added but not retained

Manufacturing Procedure:

The seal coated cores made in Example 3 were further coated with a mixture of EUDRAGIT® FS 30 D:EUDRAGIT® L 30 D-55 in 85:15 (% w/w), triethyl citrate and TiO₂ dispersion in amounts shown in Table 6 as outer coating using a perforated tablet coater.

### Example 7

This example illustrates the manufacture of particulates of the present teachings.

TABLE 7

| Ingredient | mg/Dose | % |
|---|---|---|
| Example 4 coated | 685 | 100.0 |
| Outer Coat | | |
| EUDRAGIT ® FS 30D | 62 | 9.1 |
| PLASACRYL ™ T20 | 6.2 | 0.9 |
| Purified water* | q.s | — |
| Coated weight (mg) | 753.2 | — |

*Added but not retained

Manufacturing Procedure:

The seal-coated cores made in Example 4 with pectin inner coating were further coated with EUDRAGIT® FS 30D and PLASACRYL™ T20 dispersion in amounts described in Table 7. A perforated tablet coater was used to apply an outer coating and generate particulates.

### Example 8

This example illustrates the manufacture of particulates of the present teachings.

Seal-coated cores comprising a pectin inner coating are generated as in Example 4. An outer coating is then added to the seal-coated cores by spraying with EUDRAGIT® FS 30D:EUDRAGIT® 30 D—55 in 85:15 (% w/w), triethyl citrate, polysorbate 80 and TiO₂ dispersion in a perforated tablet coater.

### Example 9

This example illustrates the construction of a capsule of the present teachings.

Mesalamine cores are generated as in Example 1. These cores are then coated with pectin and talc using a perforated tablet cower. An outer coating is then added by applying a dispersion of EUDRAGIT® FS 30 D, triethyl citrate and talc using a perforated tablet coater. The particulates are then filled into gelatinous capsules.

### Example 10

This example illustrates the construction of a tablet of the present teachings.

Vancomycin cores are made as pellets in accordance with Example 2. These pellets are then coated with a layer of guar gum using a perforated tablet coater. An outer coating comprising cellulose acetate phthalate is then applied to the guar gum-coated pellets. These pellets are then mixed with microcrystal cellulose and lactose and pressed into a tablet.

### Example 11

This example illustrates disintegration of particulates according to the present teachings.

Particulates equivalent to one dose of Metronidazole for particulates from each of examples 5-7 were immersed into 10 mL of various pH buffers and observed for disintegration time (DT). Results are recorded in Table 8.

TABLE 8

DT Time (min)

| Example | pH 1.2 | pH 5.5 | pH 6.0 | pH 7.0 |
|---|---|---|---|---|
| 5 | no DT | no DT | no DT | 5 |
| 6 | no DT | 10 | 10 | 7 |
| 7 | no DT | no DT | no DT | 10 |

Particulates described in Examples 5 and 6 had an inner coating comprising HPMC (SPECTRABLEND™ White) while particulates described in Example 7 had an inner coating comprising pectin. Particulates described in Examples 5 and 7 had an outer coating comprising polymethacrylate EUDRAGIT® FS 30 D and plasticizer PLASACRYL™. Particulates described in Example 6 had an outer coating comprising a mixture of polymethacrylates EUDRAGIT® FS 30 D and EUDRAGIT® 30 D-55, and plasticizer triethylcitrate. Particulates of Examples 5 and 7 did not dissolve at pH 6.0 or below. The presence of pectin in the inner coating of particulates (Example 7) doubled the disintegration time at pH 7.0 compared to particulates having an HPMC inner coating (Example 5).

### Example 12

This example illustrates the results of in vitro dissolution testing at pH 7.0.

In these experiments, dissolution testing was performed in accordance with USP apparatus 1 (basket) or apparatus 2 (paddle).

In these experiments, uncoated metronidazole cores described in Example 3 (without an inner or outer coating) dissolved in the basket apparatus approximately 70% within 15 minutes during dissolution testing at pH 7.0 (50 RPM), reaching about 72% dissolution after 30 minutes (FIG. 4).

Cores seal-coated with HPMC (SPECTRABLEND™ White) as described in Example 3 dissolved approximately 55% in 15 minutes and 65% in 30 minutes in the same conditions (FIG. 4).

Particulates of the present teachings were tested for drug release rates using the paddle apparatus.

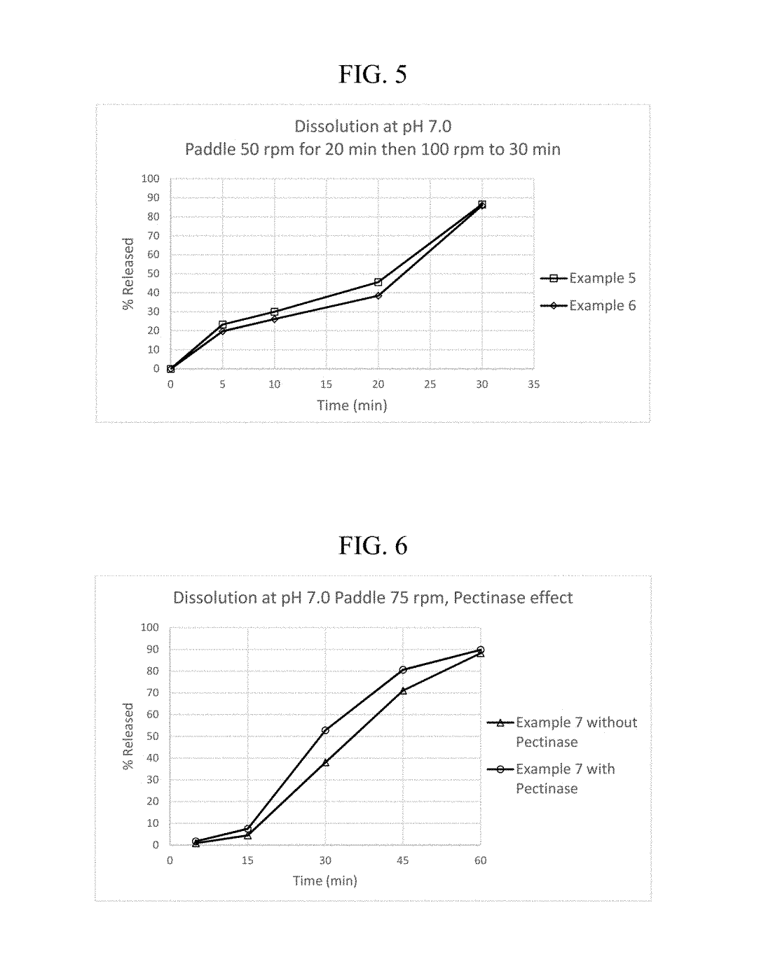

Particulates with an inner coating of HPMC and an outer coating of polymethacrylate EUDRAGIT® FS 30 D and plasticizer PLASACRYL™ (Example 5) dissolved approximately 40% in 15 minutes, and nearly 90% in 30 minutes (50 RPM for 20 minutes, 100 rpm for 30 minutes) at pH 7.0. (FIG. 5). An 85:15 ratio (% w/w) of EtlDRAGIT® FS 30 D:EUDRAGIT® L 30 D-55 (Example 6) yielded similar results (FIG. 5).

In contrast, particulates with an inner coating of pectin and an outer coating of polymethacrylate EUDRAGIT® FS 30 D and plasticizer PLASACRYL™ (Example 7) dissolved approximately 5% in 15 minutes, and >80% in 60 minutes when tested by the paddle method at 75 RPM, pH 7.0 (FIG. 6). These data indicate that a pectin inner coating can delay drug release at 15 minutes but can promote complete release by 60 minutes at pH 7.0. The particulates with pectin inner coating and polymethacrylate outer coatings of the present teaching can thus delay initial drug release prior to reaching the colon and provide complete drug release upon reaching the colon.

### Example 13

This example illustrates dissolution of particulates having a pectin inner coating in the presence or absence of pectinase.

In these experiments, dissolution testing using the paddle method (pH 7.0 phosphate buffer 75 rpm) was conducted on particulates having a pectin inner coating as described in Example 7. These particulates exhibited 40% faster dissolution in 30 minutes in the presence of pectinase (1.% v/v) compared to dissolution in the same pH 7.0 buffer in the absence of pectinase (FIG. 6). Without being limited by theory, a pectin inner coating can promote targeted release of a pharmaceutical active in the colon lumen due to the presence of pectinase from microflora.

### Example 14

This example illustrates the results of in vitro dissolution testing at pH 6.0.

Dissolution testing was performed in accordance with USP standards using apparatus 2 (paddle).

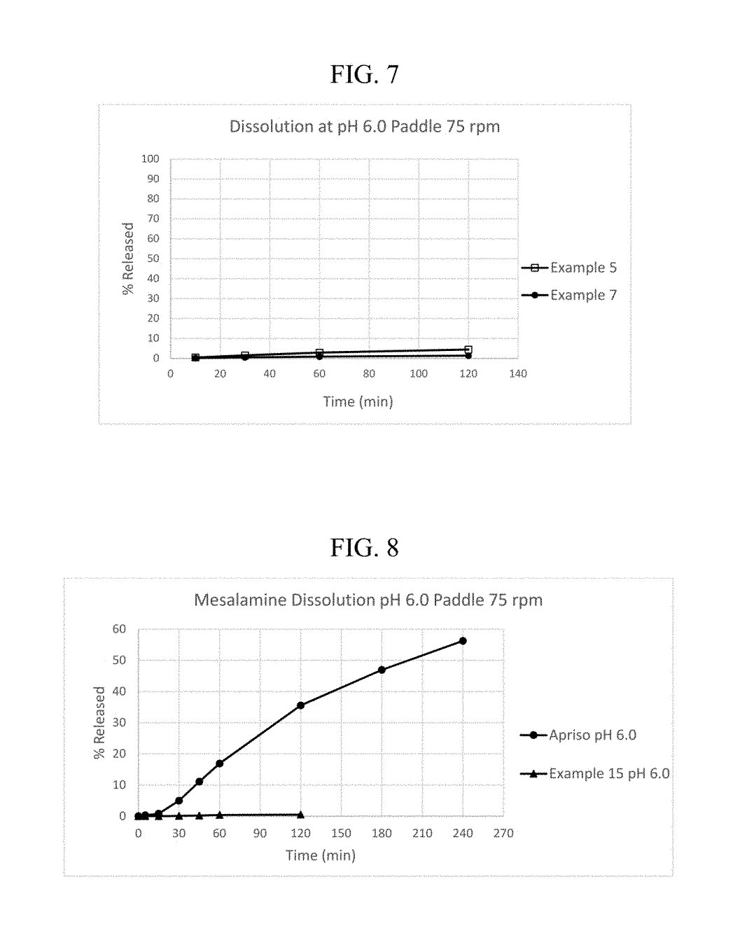

In these experiments, particulates with a EUDRAGIT® FS 30 D and PLASACRYL™ outer coating were tested for dissolution at 75 rpm paddle and pH 6.0. Under these conditions, the particulates exhibited drug release of no more than 5%, regardless of the inner coating (FIG. 7). These data illustrate that particulates of the present teachings are resistant to dissolution tinder acidic conditions such as found in the upper gastrointestinal tract.

### Example 15

This example illustrates release of a drug formulation of the present teachings in colonic conditions compared to that of a commercial formulation.

TABLE 9

| | | |
|---|---|---|
| Ingredient | mg/Dose | % |

Cores

| | | |
|---|---|---|
| Mesalamine | 375 | 57 |
| Microcrystalline cellulose PH101 | 250 | 38 |
| Hydroxypropyl cellulose EXF | 30 | 4.6 |
| Magnesium stearate | 3 | 0.5 |
| Total | 658 | 100.0 |

Inner Coating

| | | |
|---|---|---|
| Pectin | 50.6 | 7.7 |
| Glycerin | 10.2 | 1.6 |
| TiO2 | 5.0 | 0.8 |
| Purified water* | q.s. | — |
| Total weight (mg) | 723.8 | 10.0 |

Outer Coating

| | | |
|---|---|---|
| EUDRAGIT ® FS 30 D | 120.2 | 16.6 |
| PlasACRYL ™ T20 | 18.0 | 2.5 |
| Purified Water* | qs | — |
| Total weight (mg) | 862.0 | 19.1 |

In these experiments a mesalamine multiparticulate of the present teachings was manufactured with components as described in Table 9, with multiple particulates of Mesalamine cores coated with Pectin-based inner coating and EUDRAGIT®-based outer coating in 375 mg drug per dose.

The dissolution rate the mesalamine formulation was tested side-by-side with that of a currently marketed Mesalamine extended release capsule (APRISO™ (Salix Pharmaceuticals, Raleigh, N.C.), 375 mg). The inactive Ingredients of APRISO™ are listed as: colloidal silicon dioxide, magnesium stearate, microcrystalline cellulose, simethicone emulsion ethylacrylate/methylmethacrylate copolymer nonoxynol 100 dispersion, hypromellose, methacrylic acid copolymer, talc, titanium dioxide, triethyl citrate, aspartame, anhydrous citric acid, povidone, vanilla flavor, and edible black ink. In these experiments, the compositions were subjected to stirring in a USP apparatus II (paddle) at 75 rpm at 37° C. The relative dissolution rate of each drug was measured at pH 6.0 and pH 7.4. During dissolution testing at pH 6.0. the APRISO™ formulation started releasing drug as early as 30 minutes, with 36% of the drug being dissolved at the 2 hour mark; in contrast, the formulation of the present teachings that was tested did not detectably release drug for at least 2 hours at pH 6 (FIG. 8).

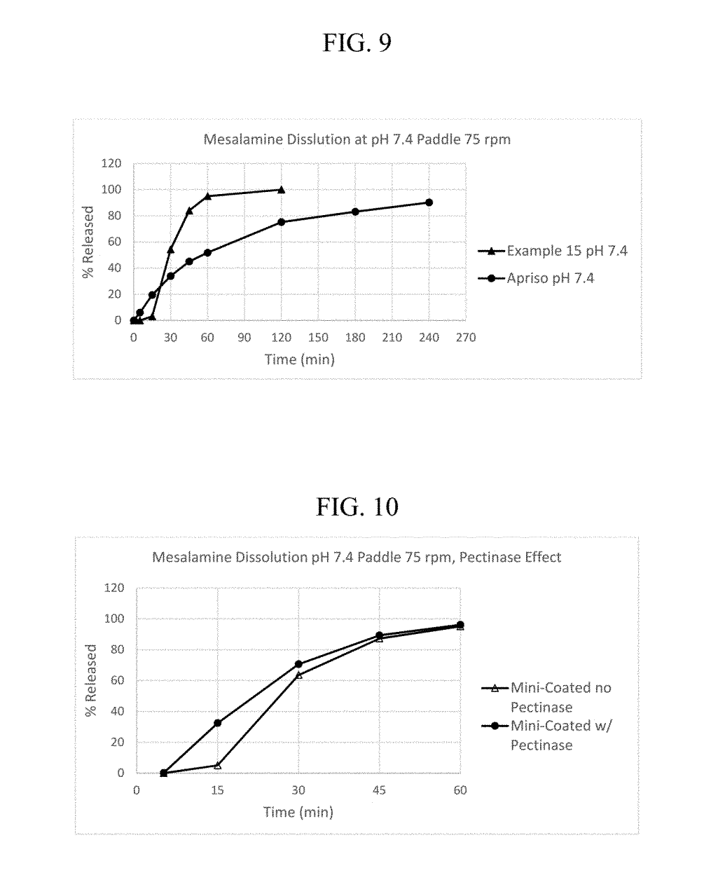

In contrast, at pH 7.4, the mesalamine formulation of the present teachings showed a complete release within 60 minutes from the beginning of the test, whereas the APRISO™ formulation showed only about 50% release in the same time, taking four hours to release 90% of the drug payload (FIG. 9).

These results illustrate that compared to a commercial formulation, a drug formulation of the present teachings can exhibit greater resistance to dissolution at stomach pH but can more rapidly dissolve at colonic pH.

### Example 16

This example illustrates that pectinase can increase the dissolution rate of formulations of the present teachings.

In these experiments, the mesalamine formulation in Example 15 was tested for dissolution in the presence and absence of pectinase. FIG. 10 illustrates that at pectinase concentration of 1.6% viv in dissolution medium at pH 7.4, mesalamine release at the 15 min time point was six times higher relative to mesalamine release in the absence of pectinase.

These data show that the dissolution rate of a drug formulation of the present teachings can be enhanced by pectinase, an enzyme present in the colonic environment.

All publications, patents, patent applications and other references cited in this application are herein incorporated by reference, each in its entirety.

What is claimed is:

1. A particulate comprising:
a core comprising a pharmaceutical compound;
an inner coating surrounding the core, wherein the inner coating comprises a pharmaceutically acceptable polysaccharide that is susceptible to enzymatic digestion by one or more enzymes present in colonic microflora; and
an outer coating surrounding the inner coating, wherein the outer coating comprises a polymer which is stable at stomach pH and upper intestine luminal pH but dissolves at colon luminal pH, wherein the particulate dissolves at colon luminal pH in less than about 60 minutes.

2. A particulate in accordance with claim 1, wherein the pharmaceutical compound is selected from the group consisting of metronidazole, mesalamine, budesonide, mesalazine, vancomycin, fidaxomicin, rifaximin, ciprofloxacin, sulfasalazine, olsalazine, balsalazide, prednisone, hydrocortisone, infliximab, adalimumab, azathioprine, esomeprazole, mercaptopurine, vedolizumab, ciprofloxacin, golimumab, loperamide, methotrexate and pantoprazole, a pharmaceutically acceptable salt thereof and a combination thereof.

3. A particulate in accordance with claim 1, wherein the pharmaceutical compound is metronidazole.

4. A particulate in accordance with claim 1, wherein the core further comprises an excipient.

5. A particulate in accordance with claim 4, wherein the excipient is selected from the group consisting of a diluent, a binder, a disintegrant, a lubricant, a glidant and a combination thereof.

6. A particulate in accordance with claim 1, wherein the polysaccharide is a pectinase substrate.

7. A particulate in accordance with claim 1, wherein the polysaccharide is selected from the group consisting of a pectin, an amylose, a guar gum, an inulin, a dextran, a chitosan, a chondroitin sulfate and a combination thereof.

8. A particulate in accordance with claim 1, wherein the polysaccharide is a pectin.

9. A particulate in accordance with claim 1, wherein the polymer which is stable at stomach pH and upper intestine luminal pH but dissolves at colon luminal pH is selected from the group consisting of a polymethacrylate, a cellulose acetate phthalate (CAP), a cellulose acetate trimellitate (CAT), a hydroxypropylmethylcellulose phthalate (HPMCP) and a combination thereof.

10. A particulate in accordance with claim 1, wherein the outer coating is stable at pH≦6 but dissolves at pH>6.

11. A particulate in accordance with claim 1, wherein the outer coating comprises a polymethacrylate.

12. A particulate in accordance with claim 11, wherein the polymethacrylate is selected from the group consisting of poly(methyl acrylate-co-methyl methacrylate-co-methacrylic acid) 7:3:1, poly(methacrylic acid-co-methyl methacrylate) 1:1, poly(methacrylic acid-co-methyl methacrylate) 1:2, poly(methacrylic acid-co-ethyl acrylate) 1:1, and a combination thereof.

13. A particulate in accordance with claim 11, wherein the polymethacrylate is poly(methyl acrylate-co-methyl methacrylate-co-methacrylic acid) 7:3:1.

14. A pharmaceutical dosage form comprising:
a plurality of particulates in accordance with claim 1; and
additional excipient material selected from the group consisting of a binder, a colorant, a disintegrant, a lubricant, a glidant, a flavoring, a preservative, a diluent and a combination thereof.

15. A pharmaceutical dosage for in accordance with claim 14, wherein the pharmaceutical dosage form is a tablet.

16. A pharmaceutical dosage form in accordance with claim 14, wherein the pharmaceutical dosage form is a capsule and further comprises a shell comprising a material selected from the group consisting of a gelatin, a hydroxypropylmethyl cellulose and a combination thereof.

17. A pharmaceutical dosage form in accordance with claim 14, wherein the pharmaceutical compound is metronidazole.

18. A pharmaceutical dosage form in accordance with claim 14, wherein the core further comprises an excipient selected from the group consisting of a diluent, a binder, a disintegrant, a lubricant, a glidant and a combination thereof.

19. A pharmaceutical dosage form in accordance with claim 14, wherein the polysaccharide is a pectin.

20. A pharmaceutical dosage form in accordance with claim 14, wherein the outer coating comprises poly(methyl acrylate-co-methyl methacrylate-co-methacrylic acid) 7:3:1.

21. A pharmaceutical dosage form in accordance with claim 15, wherein the pharmaceutical dosage form is a tablet, the pharmaceutical compound is metronidazole, the total amount of metronidazole in the tablet is from 200-800 mg, the core of each particulate further comprises an excipient selected from the group consisting of a diluent, a binder, a disintegrant, a lubricant, a glidant and a combination thereof, the inner coating comprises a pectin, and the outer coating comprises poly(methyl acrylate-co-methyl methacrylate-co-methacrylic acid) 7:3:1.

22. A pharmaceutical dosage form in accordance with claim 16, wherein the pharmaceutical dosage form is a capsule, the pharmaceutical compound is metronidazole, the total amount of metronidazole in the capsule is from 200-800 mg, the core of each particulate further comprises an excipient selected from the group consisting of a diluent, a binder, a disintegrant, a lubricant, a glidant and a combination thereof, the inner coating comprises a pectin, and the outer coating comprises poly(methyl acrylate-co-methyl methacrylate-co-methacrylic acid) 7:3:1.
